(19) United States
(12) Patent Application Publication
(10) Pub. No.: US 2007/0233255 A1
(43) Pub. Date: Oct. 4, 2007

(54) Intervertebral disc replacement prosthesis

(75) Inventors: John K. Song, Chicago, IL (US); Jeffrey David Gordon, Saratoga Springs, NY (US)

Correspondence Address: John K Song, 474 North Lakeshore Drive, #4508, Chicago, IL, 60611, US

(73) Assignee: Vermillion Technologies, LLC, Chicago, IL (US)

(21) Appl. No.: 11/714,486

(22) Filed: Mar. 6, 2007

Related U.S. Application Data
(60) Provisional application No. 60/779,354, filed on Mar. 6, 2006.

(51) Int. Cl. A61F 2/44

(52) U.S. Cl. 623/17.11

(57) ABSTRACT

An intervertebral disc replacement prosthesis for placement between two adjacent vertebrae which comprises two bone cantacting members and an intermediate member with a cavity. A first bone contacting member has bone contacting surface for contacting and/or incorporating to a first vertebra and a bearing surface which substantially mates with the intermediate member. A second bone contacting member has a bone contacting surface for contacting and/or incorporating to a second vertebra, a bearing surface which substantially mates with the intermediate bearing member, and a flexible protrusion which substantially captures the intermediate bearing member and contacts and/or incorporates with the first vertebra. The flexible member may be in the form of a spring, flexible cable, or polymer.

### CROSS-REFERENCE TO RELATED PATENT APPLICATION

This application claims priority to and the benefit of, pursuant to 35 U.S.C. § 119(e), U.S. provisional patent application Ser. No. 60/779,354, filed Mar. 6, 2006, entitled “Interververtebral disc replacement prosthesis” by John K Song and Jeffrey D. Gordon and is incorporated herein by reference in its entirety.

### FIELD OF THE INVENTION

The present invention generally relates to a prosthesis for the spine which is used to partially, or completely replace an intervertebral disc.

### BACKGROUND OF THE INVENTION

Degenerative spinal disease results from the progressive degeneration of the spinal disc (common terms are “disc bulge”, “slipped disc”, “herniated disc”) and of the articulations between the bones of the spine called the facets (resulting in painful and enlarged joints). It is a major source of disability and lost work. Symptoms of the disease are back pain caused by painful joint contact, overstrained ligaments and muscles, and numerous other factors. In addition, nerve pain (“radiculopathy”, “sciatica”) can occur from pressure on nerves from bone spurs, herniated discs, and/or narrowing of the foramen (the nerve root path through the spine).

Current treatment of degenerative spine disease can range from non-surgical methods such as physical therapy, pain medication and rest, to removal of bone spurs and/or herniated discs. Increasingly more prevalent is the use of spinal fusion whereby screws and rods are used to fix the painful joints in place. However, it is becoming recognized that while this works in the short term, in the long term the adjacent disc levels are forced to over-extend because of the loss of motion at the fused level. Subsequently, these adjacent levels degenerate more quickly and often require additional surgery.

Total Disc Arthoplasty (TDA) is the newest and most advanced area of research in spinal surgery. TDA is the replacement of the spinal disc and is intended to relieve pain while maintaining normal spinal motion to prevent adjacent discs from degenerating. In Europe, TDA has been practiced for over 20 years. A recognized benefit has been dramatically shortened patient recovery periods as compared to fusion.

As TDA is a novel technology, it must gain acceptance within the medical community. In order to do so, clinical efficacy, safety and patient satisfaction must be demonstrated. In addition, surgeon comfort with the implant and implantation procedure must be established . There are a handful of disc replacement devices in development and in clinical trials. However, examination of current technology has revealed that the devices under development do not correctly or adequately address a multitude of factors including: re-establishment of normal spinal motion, durability, simplicity, and ease of use.

### SUMMARY OF THE INVENTION

A preferred embodiment of the present invention is an intervertebral disc replacement prosthesis for placement between two adjacent vertebrae which comprises two endplates and an intermediate bearing member with a central hole. A first endplate member for contacting a first vertebra has a surface for contacting and/or incorporating to a first vertebra and a bearing surface which substantially mates with the intermediate bearing member. A second endplate member has a surface for contacting and/or incorporating to a second vertebra, a bearing surface which substantially mates with the intermediate bearing member, and a flexible protrusion which substantially captures the intermediate bearing member and contacts and/or incorporates with the first vertebra. The flexible member may be in the form of a helical slit or transverse slit flexure. A through hole is incorporated in the second endplate member with an axis substantially axial to the spring member. A flexible element, such as a braided cable, is attached to the first endplate member and the second endplate member and extends through the axial hole in the spring portion of the second endplate member. This cable is meant to act as a failsafe to prevent migration of pieces in the event of spring failure. The cable is installed so that it does not limit the motion of the intervertebral disc replacement prosthesis.

An alternative embodiment of the invention is an intervertebral disc replacement prosthesis for placement between two adjacent vertebrae which comprises two endplates and an intermediate bearing member with a central hole. A first endplate member for contacting a first vertebra has a surface for contacting and/or incorporating to a first vertebra and a bearing surface which substantially mates with the intermediate bearing member. A second endplate member has a surface for contacting and/or incorporating to a second vertebra, a bearing surface which substantially mates with the intermediate bearing member, and a flexible protrusion which substantially captures the intermediate bearing member and contacts and/or incorporates with the first vertebra. The flexible member is in the form of a spring. A blind or through hole is incorporated in the second endplate member with an axis substantially axial to the spring member.

A second alternative embodiment of the invention is an intervertebral disc replacement prosthesis for placement between two adjacent vertebrae which comprises two endplates, an intermediate bearing member with a central hole, and a flexible member. A first endplate member for contacting a first vertebra has a surface for contacting and/or incorporating to a first vertebra and a bearing surface which substantially mates with the intermediate bearing member. A second endplate member has a surface for contacting and/or incorporating to a second vertebra and a bearing surface which substantially mates with the intermediate bearing member. A through hole is incorporated in the first and second endplate members with an axis approximately parallel to the axis of the hole through the intermediate bearing member and approximately parallel to the axis of the spinal column. A flexible member, such as a braided cable, is introduced within the holes in the first and second endplate members and the hole in the intermediate bearing member and may be rigidly connected to said endplate members or at least is in contact with said endplate members. The flexible member is meant to capture the intermediate bearing member and may also act to limit motion of said first endplate member relative to said second endplate member.

A further alternative embodiment of the invention is an intervertebral disc replacement prosthesis for placement between two adjacent vertebrae which substantially resembles the second alternative embodiment where there are more than one intervertebral disc replacement prosthesis to be incorporated within a single disc space.

It is an object of the present invention to provide an apparatus for placement between two adjacent vertebrae which acts to mimic the motion of a normal intervertebral disc.

It is another object of the present invention to provide an apparatus for placement between two adjacent vertebrae which acts to mimic the stiffness of a normal intervertebral disc.

It is another object of the present invention to provide an apparatus for placement between two adjacent vertebrae which is substantially contained and connected so as to be a single unit. The advantages of such an apparatus include ease of surgical placement of the apparatus and prevention of migration of one or more portions of the apparatus from the surgically implanted site.

It is another object of the present invention to provide multiple apparatuses for placement between two adjacent vertebrae which can be implanted though small openings such as occurs during implantation into a disc space from a posterior direction through spinal and neurological structures.

### BRIEF DESCRIPTION OF THE DRAWINGS

FIG. 1a is a perspective view of the preferred embodiment of the invention

FIG. 1b is a sectioned view of the preferred embodiment of the invention

FIG. 1c is an exploded, perspective view of the preferred embodiment of the invention showing the assembly

FIG. 2a is a perspective view of an alternative embodiment of the invention

FIG. 2b is a detail view FIG. 2a

FIG. 2c is a sectioned view of an alternative embodiment of the invention

FIG. 2d is an exploded, perspective view of an alternative embodiment of the invention showing the assembly

FIG. 2e is a detail view FIG. 2d

FIG. 3a is a perspective view of a second alternative embodiment of the invention

FIG. 3b is an exploded, perspective view of a second alternative embodiment of the invention showing the assembly

FIG. 3c is a sectioned view of a second alternative embodiment of the invention

FIG. 4a is a perspective view of a third alternative embodiment of the invention showing two apparatuses for adjacent implantation

FIG. 4b is a perspective view of a third alternative embodiment of the invention showing a single apparatus

FIG. 4c is an exploded, perspective view of a third alternative embodiment of the invention showing the assembly

FIG. 4d is a reverse, perspective view of the intermediate bearing member

### DETAILED DESCRIPTION OF THE DRAWINGS

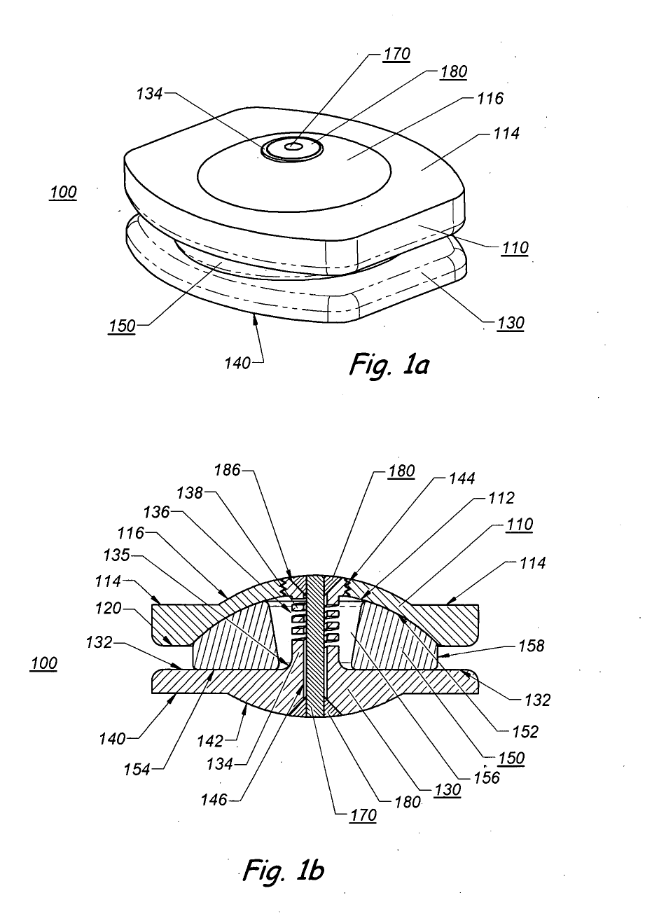

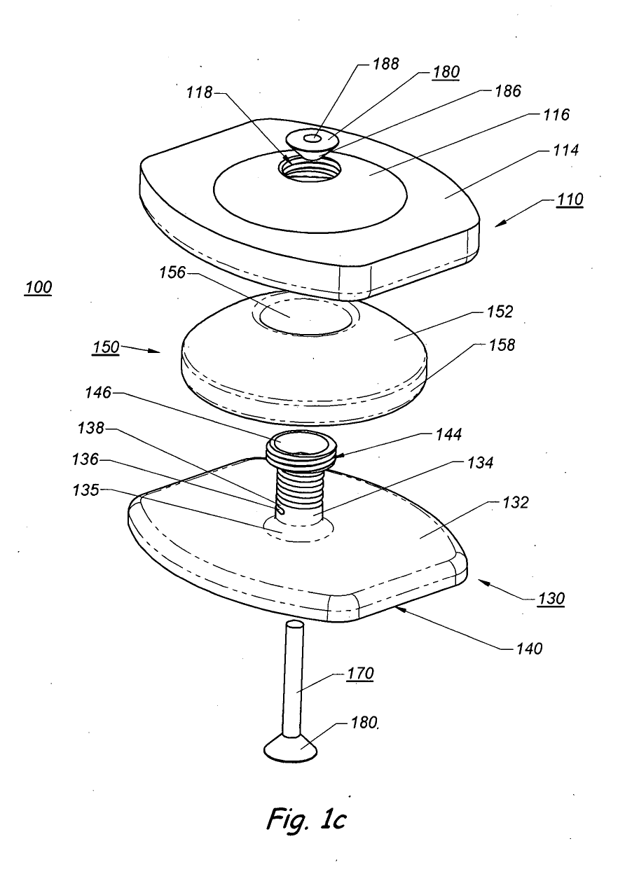

Pictured in FIGS. 1a, 1b and 1c is a preferred embodiment of the invention. A first bone contacting member 110 includes a bone contacting surface 114, a boss 116 a threaded hole 118, and a bearing surface 112. A second bone contacting member 130 includes a bone contacting surface 140, a boss 142, a bearing surface 132, protrusion 134 which has a cavity 146, a threaded end 144, and a helical slit 136 creating a helical spring 138. Protrusion 134 therefore is a flexible member which can flex about the axes perpendicular to the axis of cavity 146. An intermediate member 150 includes a first bearing surface 152, a second bearing surface 154 and a cavity 156 which may be tapered. A flexible member 170 has endpieces 180 which may be crimped, swaged, welded, soldered, bonded or otherwise rigidly connected to flexible member 170. The assembly of the preferred embodiment of the present invention, illustrated by FIG. 1c involves passing intermediate member 150 over protrusion 134 of second bone contacting member 130 until bearing surface 154 of intermediate member, 150 contacts bearing surface 132 of second bone contacting member 130. First bone contacting member 110 is threaded onto threaded end 144 of second bone contacting member 130. This connection is shown as a threaded connection, but could also be a welded, soldered, or pinned connection as well as utilizing a retaining ring, a spring clip or other connecting means. The connection could also include any combination of these methods. Flexible member 170 has an endpiece 180 connected as stated above and is passed though through cavity 146 and a second endpiece 180 is attached to flexible element 170.

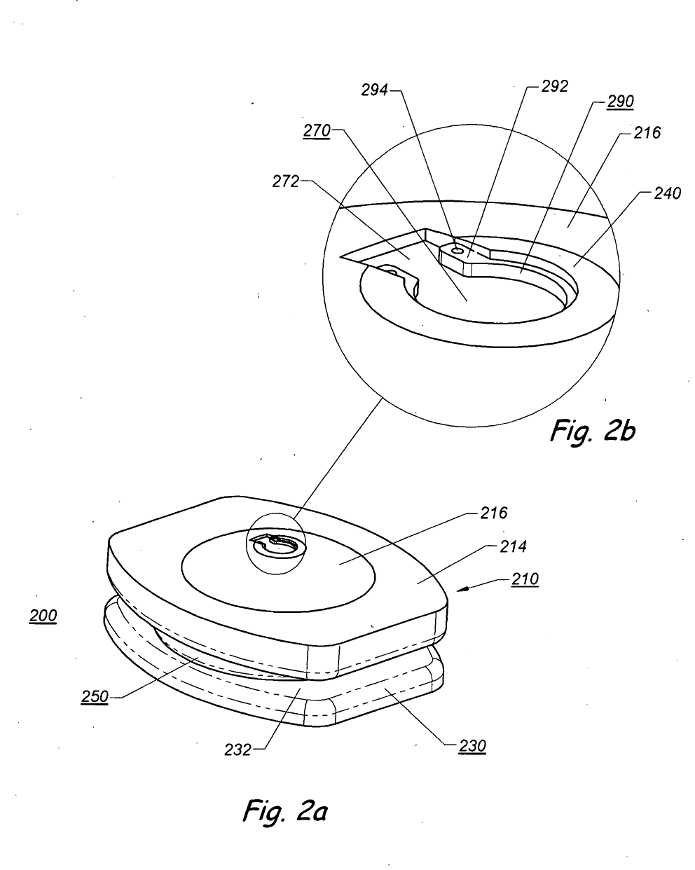

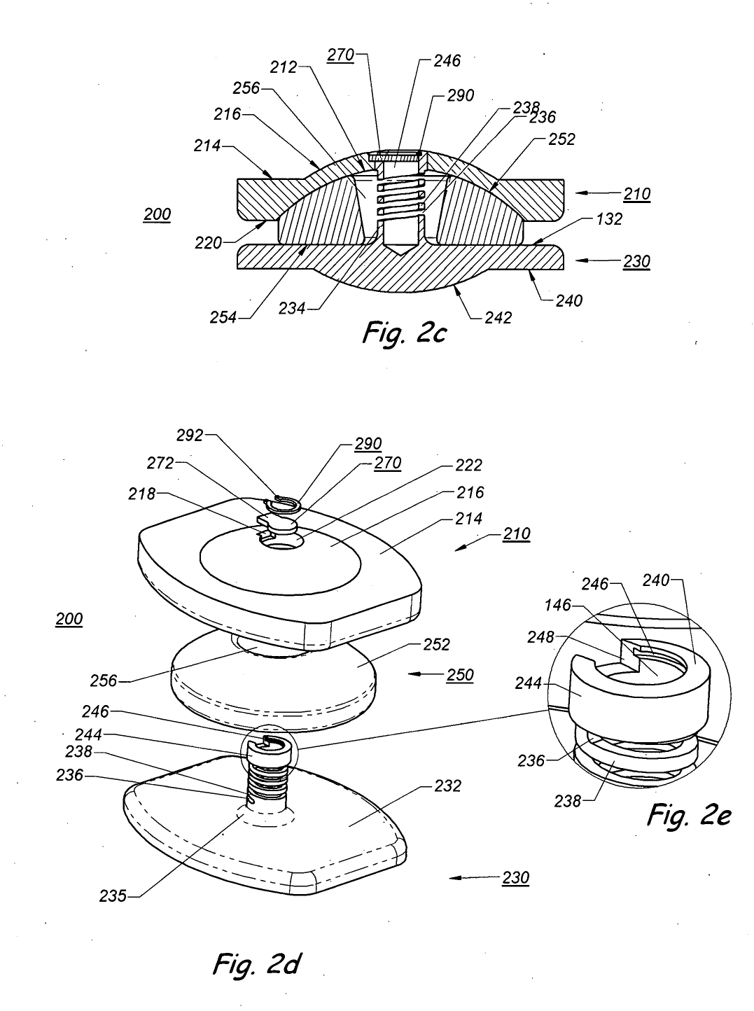

Pictured in FIGS. 2a, 2b, 2c, 2d and 2e is an alternative embodiment of the invention. A first bone contacting member 210 includes a bone contacting surface 214, a boss 216 a cavity 222, a keyway 218, and a bearing surface 212. A second bone contacting member 230 includes a bone contacting surface 240, a boss 242, a bearing surface 232, an protrusion 234 which has a cavity 246, a connection boss 244 and a helical slit 236 creating a helical spring 238. Protrusion 134 therefore is a flexible member which can flex about at least the two axes perpendicular to cavity 246. Connection boss 244 includes a retaining ring groove 247, a slot 248, and a second bone contacting surface 240 as illustrated in detail view FIG. 2e. An intermediate member 250 includes a first bearing surface 252, a second bearing surface 254 and a cavity 256 which may be tapered. FIG. 2d illustrates the assembly of the alternative embodiment of the present invention. Intermediate member 250 is passed over protrusion 234 of second bone contacting member 230 until bearing surface 254 of intermediate member 250 contacts bearing surface 232 of second bone contacting member 230. First bone contacting member 210 is slid over or pressed onto connection boss 244 of second bone contacting member 230. To prevent rotation of first bone contacting member 210 about the axis of connection boss 244 of second bone contacting member 230, a key 270 with a flange 272 is introduced into cavity 246 so that flange 272 fits in slot 248. A retaining ring 290 is compressed with a tool (not pictured) and introduced into retaining ring groove 247 and released so that it is captured in retaining ring groove 247. This connection could also be a welded, soldered, threaded, or pinned connection as well as utilizing a spring clip or other connecting means. The connection could also include any combination of these methods.

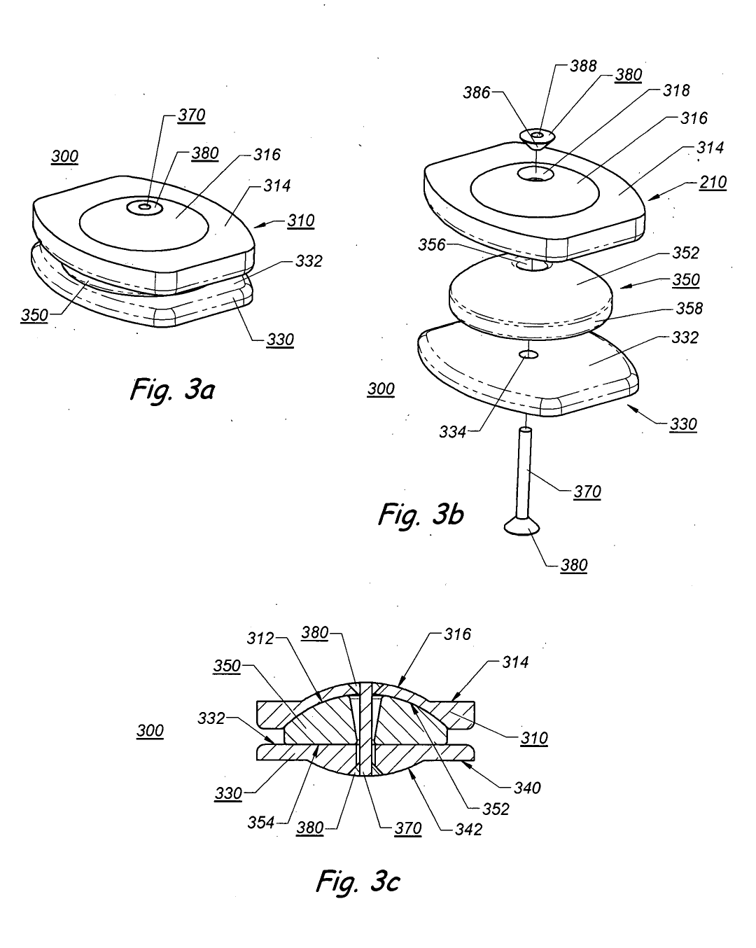

Pictured in FIGS. 3a, 3b, and 3c is a second alternative embodiment of the invention. A first bone contacting member 310 includes a bone contacting surface 314, a boss 316 a cavity 318, and a bearing surface 312. A second bone contacting member 330 includes a bone contacting surface 340, a boss 342, a bearing surface 332, and a cavity 334. An intermediate member 350 includes a first bearing surface 352, a second bearing surface 354 and a cavity 356 which may be tapered. A flexible member 370 has endpieces 380 which may be crimped, swaged, welded, soldered, bonded or otherwise rigidly connected to cable 370. The assembly of the second alternative embodiment of the present invention, illustrated by FIG. 3b involves bringing into contact second bearing surface 354 of intermediate member 350 with bearing surface 332 of second bone contacting member 330 so that the axis of cavity 356 of intermediate member 350 is approximately aligned with the axis of cavirty 334 of second bone contacting member 330. Bearing surface 312 of first bone contacting member 310 is brought into contact with first bearing surface 352 of intermediate member 350 so that the axis of cavity 356 of intermediate member 350 is approximately aligned with the axis of cavity 318 of first bone contacting member 310. A flexible member 370 has an end piece 380 connected as stated above and is passed though through cavity 334, through cavity 356, and cavity 318 and a second end piece 180 is attached to flexible member 170.

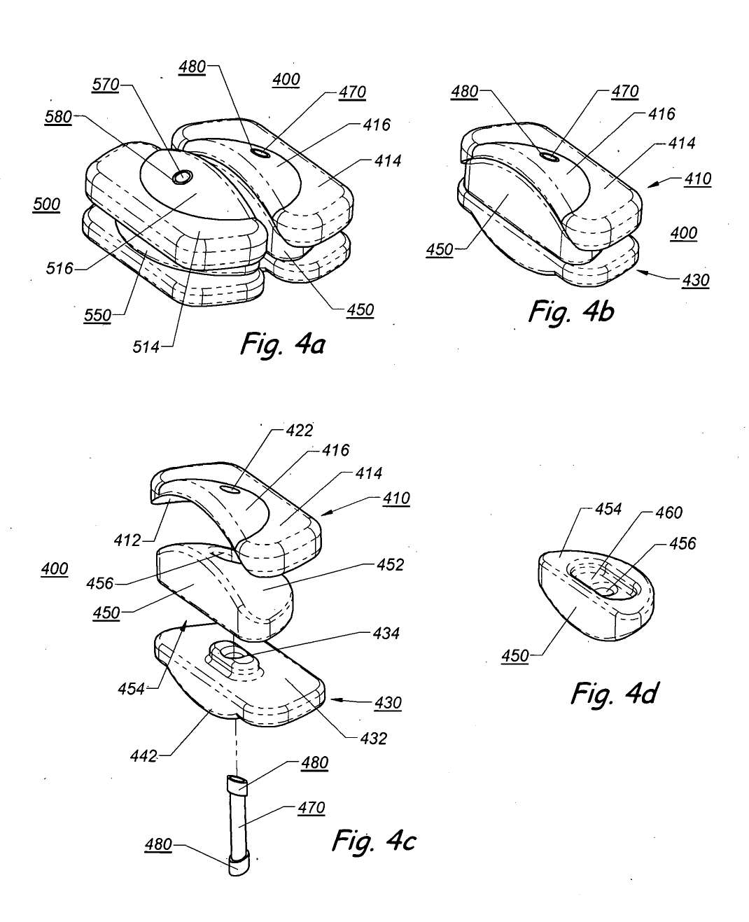

Pictured in FIGS. 4a through 4d is a third alternative embodiment of the invention. A first bone contacting member 410 includes a bone contacting surface 414, a boss 416 a cavity 422, and a bearing surface 412. A second bone contacting member 430 includes a bone contacting surface 440, a first boss 442, a bearing surface 432, a second boss 436, and a cavity 446. An intermediate member 450 includes a first bearing surface 452, a second bearing surface 454, a slot 460 and a cavity 456 which may be tapered. A flexible member 470 has endpieces 480 which may be crimped, swaged, welded, soldered, bonded or otherwise rigidly connected to cable 470. A second device is shown in FIG. 4a which further includes a third bone contacting member 510 which consists of a bone contacting surface 514, a boss 516 a cavity 522, and a bearing surface 512. A fourth bone contacting member 530 includes a bone contacting surface 540, a boss 542, a bearing surface 532, and a cavity 546. A second intermediate member 550 includes a first bearing surface 552, a second bearing surface 554 and a cavity 556 which may be tapered. FIG. 4c illustrates the assembly of one device of the third alternative embodiment of the present invention. Second bearing surface 454 of intermediate member 450 is brought into contact with bearing surface 432 of second bone contacting member 430 so that the axis of cavity 456 of intermediate member 450 is approximately aligned with the axis of cavity 434 of second bone contacting member 430. Second boss 436 fits into slot 460 to prevent or substantially limit rotation of intermediate member 450 relative to bone contacting member 430. Bearing surface 412 of first bone contacting member 410 is brought into contact with first bearing surface 452 of intermediate member 450 so that the axis of cavity 456 of intermediate member 450 is approximately aligned with the axis of cavity 422 of first bone contacting member 410. A flexible member 470 has an endpiece 480 connected as stated above and is passed though cavity 434, through cavity 456 and cavity 418 and a second endpiece 480 is attached to flexible member 470. FIG. 4d is a reverse view of intermediate member 450 showing slot 460.

What is claimed is:

1. An implantable prosthesis, comprising:
a. a first bone contacting member, a second bone contacting member, and an intermediate member;
b. said first bone contacting member comprising a bone contacting surface for contacting a first vertebra, and a bearing surface for articulating with said intermediate member;
c. said second bone contacting member comprising: a first bone contacting surface for contacting a second vertebra adjacent to said first vertebra, a bearing surface for articulating with said intermediate member, a protrusion comprising:
i. an axis, a cavity therein along said axis, a perimeter surface, and at least one slit defined in said perimeter surface to provide flexibility to said protrusion,
ii. a second bone contacting surface for contacting said first vertebra
d. said intermediate member comprising a first bearing surface for articulation with said first bone contacting member, a second bearing surface for articulation with said second bone contacting member, and a cavity therein connecting said first and second bearing surfaces,
e. said prosthesis assembled such that said protrusion of said second bone contacting member is contained substantially within said cavity of said intermediate member, and said first bone contacting member is substantially connected to said second bone contacting member.

2. The prosthesis of claim 1, wherein the first bearing surface of the intermediate member has a convex surface, and the bearing surface of the first bone contacting member has a concave surface suitable for receiving and communicating with the convex surface of the intermediate member.

3. The prosthesis of claim 1, wherein the second bearing surface of the intermediate member has a convex surface, and the bearing surface of the second bone contacting member has a concave surface suitable for receiving and communicating with the convex surface of the intermediate member.

4. The prosthesis of claim 1, wherein the bearing surface of the second bone contacting member and the second bearing surface of the intermediate member are both substantially planar.

5. The prosthesis of claim 1, wherein the bearing surface of the first bone contacting member and the first bearing surface of the intermediate member are both substantially planar.

6. The prosthesis of claim 1, wherein the at least one slit defined in the protrusion of the second bone contacting member is helical.

7. The prosthesis of claim 1, wherein the at least one slit defined in the protrusion of the second bone contacting member is transverse.

8. The prosthesis of claim 1, wherein the bearing surfaces of either said first or second bone contacting members contains at least one protrusion for the purpose of reducing or eliminating rotation between that member and the intermediate member.

9. The prosthesis of claim 1, further comprising a flexible member, said flexible member having an axis and being flexible at least about the two axes perpendicular to said axis; said flexible member contacting said first and second bone contacting members and being housed with said cavity of said protrusion of said second bone contacting member.

10. The prosthesis of claim 9, wherein the flexible member is wound or braided cable.

11. The prosthesis of claim 9, wherein the flexible member is a spring.

12. The prosthesis of claim 9, wherein the flexible member is a polymer.

13. An implantable prosthesis, comprising:
a. a first bone contacting member, a second bone contacting member, an intermediate member, and a flexible member;
b. said first bone contacting member comprising a bone contacting surface for contacting a first vertebra, and a bearing surface for articulating with said intermediate member, an axis, and a cavity along said axis;
c. said second bone contacting member comprising: a first bone contacting surface for contacting a second vertebra adjacent to said first vertebra, a bearing surface for articulating with said intermediate member, an axis, and a cavity along said axis,
d. said intermediate member comprising a first bearing surface for articulation with said first bone contacting member, a second bearing surface for articulation with said second bone contacting member, an axis, and a cavity along said axis connecting said first and second bearing surfaces,
e. said flexible member having an axis and being flexible at least about the two axes perpendicular to said axis;
f. said prosthesis assembled such that said flexible member is contained substantially within said cavity of said intermediate member, and said flexible member being substantially connected to said first and second bone contacting members.

14. The prosthesis of claim 13, wherein the bearing surfaces of either said first or second bone contacting members contains at least one protrusion for the purpose of reducing or eliminating rotation between that member and the intermediate member.

15. The prosthesis of claim 13, wherein the flexible member is wound or braided cable.

16. The prosthesis of claim l3, wherein the flexible member is a spring.

17. The prosthesis of claim 13, wherein the flexible member is a polymer.
